(19) United States
(12) Patent Application Publication
(10) Pub. No.: US 2007/0203163 A1
(43) Pub. Date: Aug. 30, 2007

(54) Novel Treatment Of Irritable Bowel Syndrome 1

(75) Inventors: Erik Lindström, Molndal (SE); Hakan Larsson, Molndal (SE); Anders Lehmann, Molndal (SE)

Correspondence Address: BIRCH, STEWART, KOLASCH & BIRCH, LLP, P.O. BOX 747, 8110 GATEHOUSE ROAD, SUITE 500 EAST, FALLS CHURCH, VA, 22040-0747, US

(73) Assignees: ASTRAZENECA AB, SODERTALJE (SE); NPS PHARMACEUTICALS, INC., SALT LAKE CITY, UT (US)

(21) Appl. No.: 10/582,388

(22) PCT Filed: Dec. 8, 2004
(86) PCT No.: PCT/US04/41005

§ 371 Date: Apr. 17, 2007

(30) Foreign Application Priority Data
Dec. 17, 2003 (SE) 0303418-8

(51) Int. Cl. A61K 31/4439; A61K 31/44

(52) U.S. Cl. 514/277; 514/341

(57) ABSTRACT

The present invention relates to the use of metabotropic glutamate receptor 5 (mGluR5) antagonists for the treatment of irritable bowel syndrome (IBS).

### FIELD OF THE INVENTION

The present invention relates to the use of metabotropic glutamate receptor 5 (mGluR5) antagonists for the treatment of irritable bowel syndrome (IBS).

### BACKGROUND OF THE INVENTION

Irritable bowel syndrome (IBS) can be defined in accordance with Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II: Functional Gastrointestinal Disorders: Diagnosis, Pathophysiology and Treatment. 2 ed. McLean, V A: Degnon Associates, Inc.; 2000:351-432 and Drossman D A, Corazziari E, Talley N J, Thompson W G and Whitehead W E. Rome II: A multinational consensus document on Functional Gastrointestinal Disorders. Gut 45(Suppl.2), II1-II81.9-1-1999.

The etiology of this condition is unknown. There appears to be multiple factors that play a role in the pathogenesis of IBS including psychosocial disorders, genetic factors, dysmotility, post-acute gastrointestinal infection, abnormal brain-gut axis interactions and visceral hypersensitivity.

Currently existing therapy of IBS is largely empirical and directed towards relief of prominent symptoms. The most commonly used therapies still include bulking agents, antispasmodics and antidepressants.

The metabotropic glutamate receptors (mGluR) are G-protein coupled receptors that are involved in the regulation and activity of many synapses in the central nervous system (CNS). Eight metabotropic glutamate receptor subtypes have been identified and are subdivided into three groups based on sequence sirmilarity. Group I consists of mGluR1 and mGluR5. These receptors activate phospholipase C and increase neuronal excitability. Group II, consisting of mGluR2 and mGluR3 as well as group III, consisting of mGluR4, mGluR6, mGluR7 and mGluR8 are capable of inhibiting adenylyl cyclase activity and reduce synaptic transmission. Several of the receptors also exist in various isoforms, occurring by alternative splicing (Chen, C-Y et al., Journal of Physiology (2002), 538.3, pp. 773-786; Pin, J-P et al., European Journal of Pharmacology (1999), 375, pp. 277-294; Bräuner-Osborne, H et al. Journal of Medicinal Chemistry (2000), 43, pp. 2609-2645; Schoepp, D. D, Jane D. E. Monn J. A. Neuropharmacology (1999), 38, pp. 1431-1476).

The object of the present invention was to find a new way for the treatment of IBS.

### OUTLINE OF THE INVENTION

It has now surprisingly been found that metabotropic glutamate receptor 5 (mGluR5) antagonists are useful for the treatment of IBS.

Consequently, the present invention is directed to the use of a metabotropic glutamate receptor 5 antagonist for the manufacture of a medicament for the treatment of IBS.

A further aspect of the invention is the use of a metabotropic glutamate receptor 5 antagonist for the manufacture of a medicament for the treatment of diarrhea predominant IBS. A further aspect of the invention is a method for the treatment of diarrhea predominant IBS, whereby an effective amount of a metabotropic glutamate receptor 5 antagonist is administered to a subject suffering from said condition.

A further aspect of the invention is the use of a metabotropic glutamate receptor 5 antagonist for the manufacture of a medicament for the treatment of constipation predominant IBS. A further aspect of the invention is a method for the treatment of constipation predominant IIBS, whereby an effective amount of a metabotropic glutamate receptor 5 antagonist is administered to a subject suffering from said condition.

A further aspect of the invention is the use of a metabotropic glutamate receptor 5 antagonist for the manufacture of a medicament for the treatment of constipation. A further aspect of the invention is a method for the treatment of constipation, whereby an effective amount of a metabotropic glutamate receptor 5 antagonist is administered to a subject suffering from said condition.

A further embodiment is the use of a metabotropic glutamate receptor 5 antagonist, for the manufacture of a medicament for the inhibition of alternating bowel movements. Another aspect of the invention is a method for the inhibition of alternating bowel movements, whereby an effective amount of a metabotropic glutamate receptor 5 antagonist is administered to a subject in need of such inhibition.

A further embodiment is the use of a metabotropic glutamate receptor 5 antagonist, for the manufacture of a medicament for the inhibition of alternating bowel movement predominant IBS. Another aspect of the invention is a method for the inhibition of alternating bowel movement predominant IBS, whereby an effective amount of a metabotropic glutamate receptor 5 antagonist is administered to a subject in need of such inhibition.

IBS is herein defmed as a chronic functional disorder with specific symptoms that include continuous or recurrent abdominal pain and discomfort accompanied by altered bowel function, often with abdominal bloating and abdominal distension. It is generally divided into 3 subgroups according to the predominant bowel pattern:
1—diarrhea predominant
2—constipation predominant
3—alternating bowel movements.

Abdominal pain or discomfort is the hallmark of IBS and is present in the three subgroups. IBS symptoms have been categorized according to the Rome criteria and subsequently modified to the Rome II criteria. This conformity in describing the symptoms of IBS has helped to achieve consensus in designing and evaluating IDS clinical studies.

The Rome II diagnostic criteria are:
1—Presence of abdominal pain or discomfort for at least 12 weeks (not necessarily consecutively) out of the preceding year
2—Two or more of the following symptoms:
   a) Relief with defecation
   b) Onset associated with change in stool frequency
   c) Onset associated with change in stool consistency

For the purpose of this invention, the term “antagonist” should be understood as including full antagonists, inverse agonists, non-competitive antagonists or competitive antagonists, as well as partial antagonists, whereby a “partial antagonist” should be understood as a compound capable of partially, but not fully, in-activating the metabotropic glutamate receptor 5.

The present invention is directed to the use of any mGluR5 antagonist which has a therapeutic effect in IBS.

The term “therapy” and/or “treatment” also includes “prophylaxis” unless there are specific indications to the contrary. The terms “therapeutic” and “therapeutically” should be construed accordingly.

The term “therapeutic effect” is defined herein as an effect favourable in the context of the therapy and/or treatment of IBS.

One example of a compound having antagonistic affinity to metabotropic glutamate receptor 5, thereby being useful in accordance with the invention, is the compound 2-methyl-6-(phenylethynyl)-pyridine (often abbreviated MPEP). MPEP is commercially available from e.g. Tocris, or may be synthesized according to well-known procedures such as disclosed by K. Sonogashira et al. in Tetrahedron Lett. (1975), 50, 4467-4470.

Pharmaceutical Formulations

For clinical use, the metabotropic glutanate receptor 5 antagonists are in accordance with the present invention suitably formulated into pharmaceutical formulations for oral administration. Also rectal, parenteral or any other route of administration may be contemplated to the skilled man in the art of formulations. Thus, the metabotropic glutamate receptor 5 antagonists are formulated with at least one pharmaceutically and pharmacologically acceptable carrier or adjuvant. The carrier may be in the form of a solid, semi-solid or liquid diluent.

In the preparation of oral pharmaceutical formulations in accordance with the invention, the metabotropic glutamate receptor 5 antagonist(s) to be formulated is mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or compressed into tablets.

Soft gelatine capsules may be prepared with capsules containing a mixture of the active compound or compounds of the invention, vegetable oil, fat, or other suitable vehicle for soft gelatine capsules. Hard gelatine capsules may contain the active compound in combination with solid powdered ingredients such as lactose, saccharose, sorbitol, mannitol, potato starch, corn starch, amylopectin, cellulose derivatives or gelatine.

Dosage units for rectal administration may be prepared (i) in the form of suppositories which contain the active substance(s) mixed with a neutral fat base; (ii) in the form of a gelatine rectal capsule which contains the active substance in a mixture with a vegetable oil, paraffm oil, or other suitable vehicle for gelatine rectal capsules; (iii) in the form of a ready-made micro enema; or (iv) in the form.of a dry micro enema formulation to be reconstituted in a suitable solvent just prior to administration.

Liquid preparations for oral administration may be prepared in the form of syrups or suspensions, e.g. solutions or suspensions, containing the active compound and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of a compound of the invention in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

In one aspect of the present invention, the metabotropic glutamate receptor 5 antagonists may be administered once or twice daily, depending on the severity of the patient's condition.

Biological Evaluation

In this study the effect of acute administration of a compound on the visceromotor response to isobaric colorectal distension in rats is investigated (Ritchie J. Pain from distension of the pelvic colon by inflating a balloon in the irritable bowel syndrome. Gut 1973; 6: 105-112. Ness T J, Gebhart G F. Colorectal distension as a noxious visceral stimulus: physiological and pharmacological characterization of pseudoeffective reflexes in the rat. Brain Research 1988; 450: 153-169).

Methods

Colorectal distension (CRD) is performed in all rats (Sprague Dawley) using a paradigm of 12 consecutive distensions (or pulses) at 80 mmHg for 30 seconds each with 4.5 minute intervals (12×80 mmHg). The visceromotor respbnse is determined by quantifying phasic changes in the balloon pressure, which is processed by specially designed computer software. The compound is dissolved in saline and administered at the doses of 1, 3 and 10 μmol/kg. The compound is given intravenously in a volume of 1 mL/kg between the third and fourth distension.

Results

MPEP

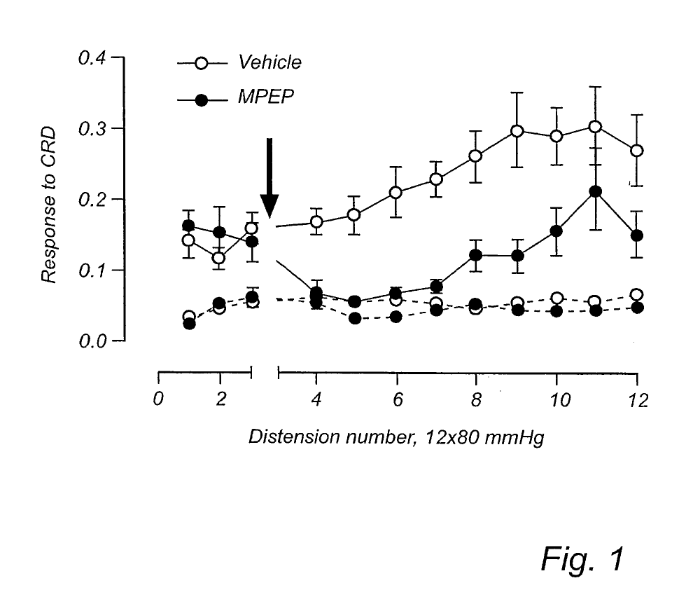

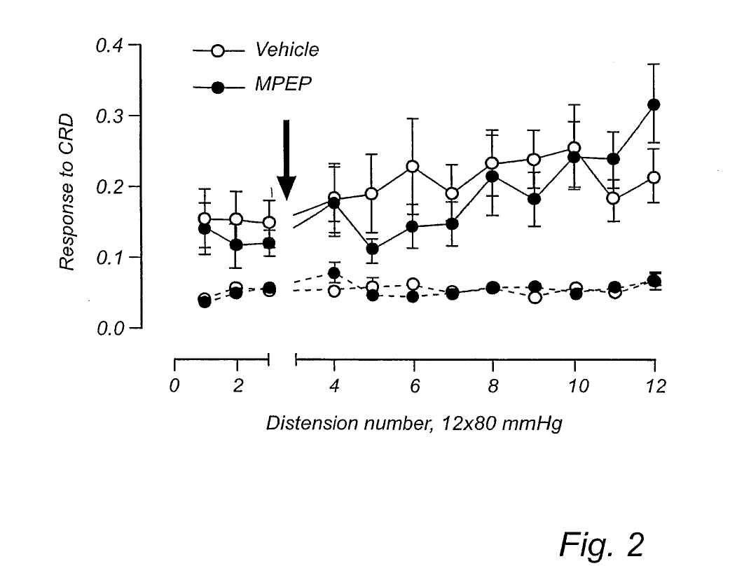

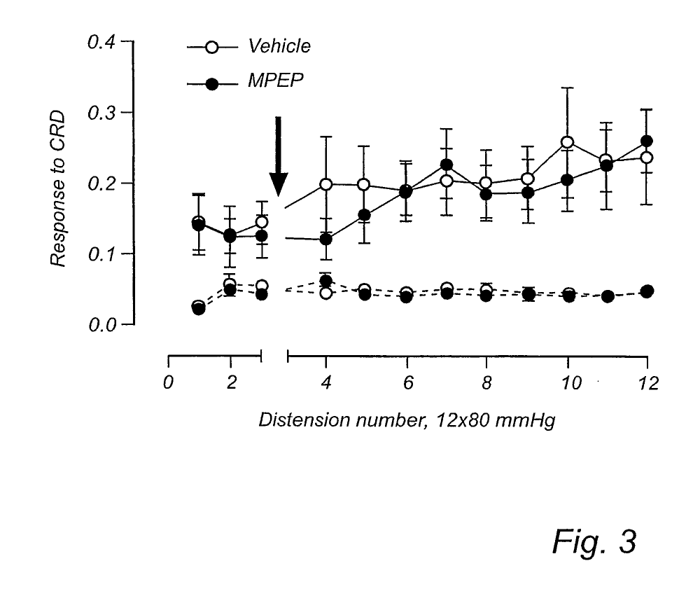

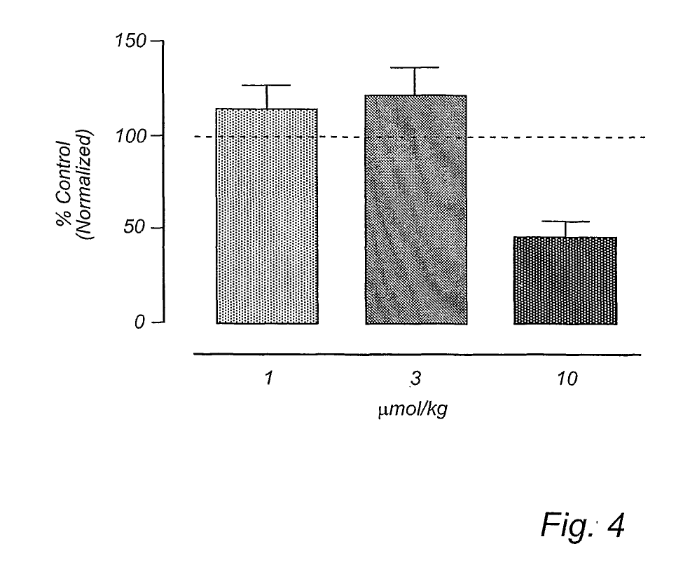

The metabotropic glutamate receptor subtype 5 (mGluR5) antagonist 2-Methyl-6-(phenylethynyl)-pyridine (MPEP) decreased the visceromotor response (VMR) when administered (10 μmol/kg) i.v during colorectal distension (FIG. 1). The average response in MPEP-treated rats was less than half of the response seen in vehicle treated (46±11% vs. 100±19, FIG. 1). 3 μmol/kg had a modest effect during a few distensions only (FIG. 2). No effect was present at a dose of 1 μmol/kg (FIG. 3). FIG. 4 summarizes the dose-dependent effect of MPEP. In all analyses data is shown as the mean±standard error of the mean, n=8. The dotted lines in the figures represent the baseline response, measured during 30 s before each distension.

3-[3-(5-Fluoro-pyridin-2-yl)-[1,2,4]oxadiazol-5-yl]-5-methoxymethyl-benzonitrile

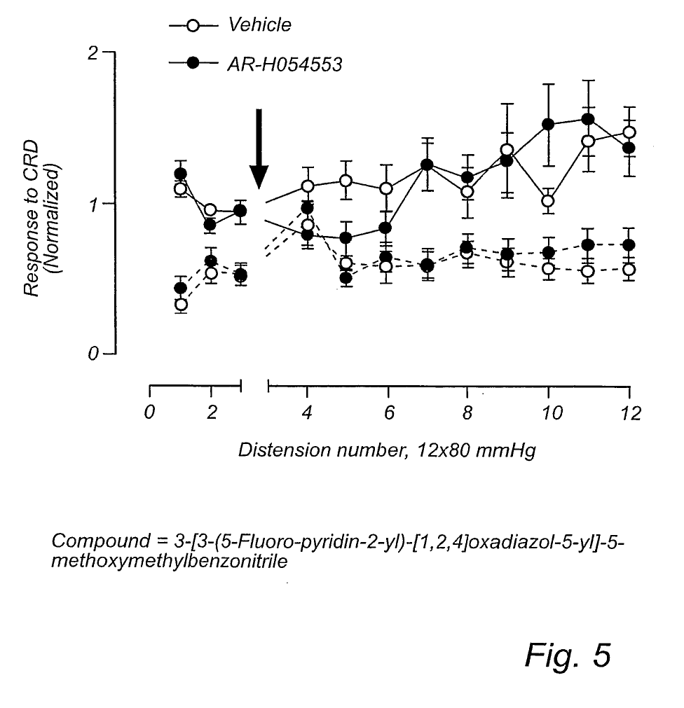

The metabotropic glutamate receptor subtype 5 (mGluR5) antagonist 3-[3-(5-Fluoro-pyridin-2-yl)-[1,2,4]oxadiazol-5-yl]-5-methoxymethyl-benzonitrile (1 μmol/kg), prepared according to the procedure described in WO02/068417, produced a transient inhibitory effect on the VMR to CRD in rats (FIG. 5).

In conclusion, antagonism of the mGluR5 receptor reduces the visceromotor response to colorectal distension.

1. (canceled)

2. (canceled)

3. (canceled)

4. (canceled)

5. (canceled)

6. (canceled)

7. (canceled)

8. A method for the treatment of irritable bowel syndrome (IBS) whereby a pharmaceutically and pharmacologically effective amount of a metabotropic glutamate receptor 5 antagonist, or a pharmaceutically acceptable salt or an optical isomer thereof, is administered to a subject in need of such treatment.

9. The method according to claim 8, wherein the IBS is diarrhea predominant IBS.

10. The method according to claim 8, wherein the IBS is constipation predominant IBS.

11. The method according to claim 8, wherein the IBS is alternating bowel movement predominant IBS.

12. A method according to any one of claims 8-11, wherein the metabotropic glutamate receptor 5 antagonist is 2-methyl-6-(phenylethynyl)-pyridine.

13. A method according to claim 12, wherein the metabotropic glutamate receptor 5 agonist is the hydrochloride salt of 2-methyl-6-(phenylethynyl)-pyridine.

14. A method according to any one of claims 8-11, wherein the metabotropic glutamate receptor 5 antagonist is 3-[3-(5-Fluoro-pyridin-2-yl)-[1,2,4]oxadiazol-5-yl]-5-methoxymethyl-benzonitrile.
